(19) United States
(12) Patent Application Publication
(10) Pub. No.: US 2025/0099925 A1
(43) Pub. Date: Mar. 27, 2025

(54) CAPSULES WITH SOLIDIFIED MATRIX

(71) Applicant: Microcaps AG, Schlieren (CH)

(72) Inventors: Rico Felber, Dietikon (CH); Morgane Roquelaure, Zurich (CH); Giulia Biffi, Zurich (CH)

(21) Appl. No.: 18/833,109

(22) PCT Filed: Feb. 3, 2023
(86) PCT No.: PCT/EP2023/052622

§ 371 Date: Jul. 25, 2024

(30) Foreign Application Priority Data
Feb. 8, 2022 (CH) CH000109/2022

(51) Int. Cl. B01F 23/41; A23P 10/35; B01F 23/70; B01F 23/80

(52) U.S. Cl. CPC B01F 23/4145; A23P 10/35; B01F 23/711; B01F 23/802

(57) ABSTRACT

Disclosed herein is a method for generating capsules with a solid matrix (7) as well as a capsule assembly comprising a plurality of capsules obtained by such a method. The method relies on temperature dependent step-emulsification of a droplet phase including a liquid hydrophobic matrix and a continuous aqueous phase.

### RELATED APPLICATIONS

This application is the National Stage entry under 35 U.S.C. 371 of International Patent Application No. PCT/EP2023/052622, filed Feb. 3, 2023, which claims priority to Swiss Patent Application No. CH000109/2022, filed Feb. 8, 2022, each of which is hereby incorporated by reference in its entirety.

### FIELD OF DISCLOSURE

The invention relates to a method for generating capsules with a solid matrix, particularly microcapsules, an assembly of capsules comprising capsules produced by this method, methods for generating a food product and a cosmetic product, the use of capsules, respectively assemblies thereof as well as a food product and a cosmetic product comprising capsules generated by the method of the invention.

### BACKGROUND, PRIOR ART

Capsules with particle sizes of 5.0 mm or less, particularly microcapsules with particle sizes of less than 1 mm, preferably of between 0.04 and 0.5 mm, have found widespread application in the field of pharmaceutics, cosmetics, diagnostics, food and material science. Such capsules may be produced from an emulsion of monodisperse droplets in a continuous phase. Monodispersity increases stability, allows to accurately control volumes in multiple chemical or biological reactions and enables the production of periodic structures, reproducible processes and ensures equal reaction conditions. Microfluidics offers an exquisite platform to precisely form monodisperse droplets. The monodisperse droplets can be cured for generating microcapsules for encapsulation of active ingredients such as drugs, fragrances, flavors, peptides, living material, such as bacteria or phages etc. fertilizers, pesticides, and other active substances for well-being.

Currently, most industrial processes for generation of microcapsules employ spray drying, high speed rotation with high shearing forces, ultrasonication, mixing and/or shaking. Noteworthy, such processes generally have the disadvantage of poor control over the capsule size, size distribution as well as surface properties of the capsules. However, these parameters are crucial for many applications. Furthermore, several compounds of interests and other components can be sensitive to the typical high shear forces and/or temperatures applied in prior art processes.

### SUMMARY OF DISCLOSURE

Hitherto known methods for producing capsules from monodisperse droplets show significant limitations. Known methods suffer from a severely limited overall operational capacity and/or from poor reproducibility and size control. However, controlling the size of the capsules is of utmost importance for various applications, particularly for applications in the pharmaceutical, fragrance and flavor industry. Furthermore, for many applications it is important to control the surface properties of the capsules, i.e. to ensure equal surface properties over each capsule and to provide even surfaces.

It is therefore a general object to advance the state of the art of generating capsules, particularly microcapsules, with a solid matrix and preferably to overcome the disadvantages of the prior art fully or partially. In favorable embodiments, a method for producing such capsules is provided allowing for accurate control of the capsule size and size distribution. In further advantageous embodiments, a method for producing such capsules is provided allowing for accurate control of the surface properties of the capsules, particularly for allowing to produce capsules with equal surface properties over each capsule and/or even surfaces. In particular embodiments, capsules formed may have a maximum difference of 1% with respect to a perfect sphere. In further particular embodiments, the coefficient of variation (CV) regarding the size distribution is below 10%, preferably below 6%. In some favorable embodiments, the surface properties of the capsules vary only by a maximum of 10%, preferably by a maximum of 5%. In further advantageous embodiments, a method for producing such capsules is provided which allows for a higher throughput and/or a more efficient production process.

The general object is achieved by the subject-matter of the independent claims. Further favorable embodiments follow from the dependent claims and the overall disclosure.

A first aspect of the invention relates to a method for generating capsules with a solid matrix, the method comprising the steps:
a. Providing in a first chamber a droplet phase at a first operating temperature. The droplet phase comprises a hydrophobic matrix. The first temperature is selected such that the hydrophobic matrix is liquid, i.e. in a liquid state of aggregation during step a. The hydrophobic matrix is configured such that it is solid at a storage temperature. The first operating temperature is higher than the storage temperature.
b. Providing in a second chamber a continuous aqueous phase at a second operating temperature, the continuous aqueous phase comprising water and optionally at least one first surfactant. In particular, the second operating temperature can be equal to or lower than the first operating temperature.

The first chamber and the second chamber are fluidic connected by one or more channels, in particular micro-channels. The method further comprises the steps:
c. Guiding the droplet phase provided in step a. from the first chamber through the one or more channels into the second chamber to form an emulsion or a dispersion of the droplet phase in the continuous aqueous phase.
d. Cooling the emulsion or dispersion of the droplet phase in the continuous aqueous phase formed in step c. below the first operating temperature such that the hydrophobic matrix solidifies to form a solid matrix thereby generating a capsule with a solid matrix, i.e. a solid hydrophobic matrix.

Although possible, it is understood that steps a. and b. must not necessarily be performed in this order. It may also be possible to first perform step b. and then step a. or perform them simultaneously.

In certain embodiments, in step d. the emulsion or dispersion of the droplet phase in the continuous aqueous phase formed in step c. is cooled down to or even below the storage temperature.

It is further understood that the dispersion or emulsion formed in step c. comprises a plurality of monodisperse droplets preferably with a particle size of 5.0 mm or less, particularly microdroplets, preferably with a particle size of 1 mm or less, comprising the droplet phase of step a. within the continuous aqueous phase of step b. as the continuous phase. It is also understood that the term “droplet phase” does not necessarily mean that this phase contains droplets, although this is possible in some embodiments, but that it is this phase which forms the droplets in step c.

It is further understood that the hydrophobic matrix is immiscible with the continuous aqueous phase. Thus, the liquid hydrophobic matrix and the continuous aqueous phase do under normal conditions (i.e. 25° C. and 1 atm.) not dissolve in each other and may therefore form a biphasic system. The hydrophobic matrix may be lipophilic. For example, the hydrophobic matrix may be an oil, a fat, a wax or mixtures thereof.

The hydrophobic matrix is configured such that it can reversibly be molten when it is heated to the first operating temperature and solidified again when it is cooled below its transition temperature, respectively the storage temperature. As used herein, the term “transition temperature” refers to the temperature below which the hydrophobic matrix solidifies and above which the hydrophobic matrix liquefies. Thus, upon cooling the temperature from a temperature above the transition temperature below the transition temperature, the hydrophobic matrix undergoes a change in its state of aggregation from liquid to solid. This process can be repeated multiple times, in particular without significantly altering and/or destroying the material, respectively the structure of the formed capsules. It is therefore clear that the first operating temperature is above the transition temperature. In certain cases, the transition temperature may not be a specific temperature value, but a temperature range, i.e. a melting range. This is in particular true if a mixture of different hydrophobic matrixes is used in the droplet phase as it may generally be the case in some embodiments of the invention.

Typically, the first operating temperature may be at least 3 K higher, in particular at least 5 K higher, in particular at least 10 K higher, in particular at least 15 K higher than the storage temperature. In certain embodiments, the first operating temperature may be in the range of 5K to 50K higher, in particular in the range of 5K to 20K higher, than the storage temperature. The term “storage temperature” can refer to a temperature being equal or lower than the transition temperature or melting point, respectively melting range, of the hydrophobic matrix. The transition temperature is the temperature at which the hydrophobic matrix undergoes a phase transition.

Typically, the liquid hydrophobic matrix is configured such that it undergoes a phase transition when it is cooled below its transition temperature. Such a transition temperature is typically between the first operating temperature and the storage temperature or equal to the storage temperature. Thus, when the formed emulsion or dispersion of the droplet phase in the continuous aqueous phase is cooled and reaches the transition temperature, a phase transition takes place and the liquid hydrophobic matrix solidifies. The solidification is a direct result of the change of temperature and is therefore typically not effected by a chemical reaction of the liquid hydrophobic matrix itself, i.e. there is for example no ion exchange reaction or the like which causes the solidification. In contrast, the solidification is a physical process being directly caused by a change of temperature as described above.

As the skilled person understands, the term “liquid” describes a nearly incompressible fluid that conforms to the shape of its container but retains a nearly constant volume independent of pressure. As used herein, a liquid may particularly have a viscosity of 10⁵ mPa·s or less, in particular of 10⁴ mPa·s or less.

One advantage of the method according to the invention is that it relies on the principles of step emulsification, i.e. the method described herein is a step emulsification method. Guiding the droplet phase of step a. through the channels enables to accurately control the size and ensures uniform size distribution of the emulsion or dispersion formed in step c. The droplets formed are optionally stabilized by the at least one first surfactant and thus their size remains essentially constant, even after the droplets are removed from the second chamber. Furthermore, the method allows a much more rapid production of capsules than the methods known in the prior art. The method disclosed herein allows for a capsule production of 100 g/h or more, or even up to 500 g/h, particularly of up to 150 kg/h or even up to 200 kg/h.

Furthermore, the method according to the invention is operationally simple as it does not rely on a chemical reaction which requires different reagents which have to be controlled such that they react at a specific moment. However, as solidification only occurs after the step emulsification took place, the advantages of step emulsification can be exploited, i.e. to first generate an emulsion or dispersion of microdroplets, whose size can be accurately controlled and only induce solidification thereafter, enabling accurate control of the size and size distribution of the formed capsules.

The formed capsules are typically massive capsules, i.e. the solid matrix is distributed over the whole capsule volume. Thus, the capsules formed do typically not comprise a liquid core at the storage temperature, in particular not a single liquid core being enclosed by a capsule wall which particularly makes up for at least 40% of the capsule volume. However, it is still possible that each capsule comprises multiple microdroplets of an aqueous phase, e.g. water. The solid matrix typically protrudes each capsule. Therefore, the capsules are solid capsules. In some embodiments, the capsules formed essentially consist of the solid matrix and optionally of the at least one compound of interest.

In some embodiments, the amount of the hydrophobic matrix in the droplet phase is at least 60 wt %, in particular at least 80 wt %.

In some embodiments, the first surfactant is selected from polyvinylalcohol (PVA), a polysorbate, such as Tween 20 (poly sorbate 20 CAS 9005-64-5) or Tween 80 (poly sorbate 80 CAS 9005-65-6), saponins, sapogenins, i.e. quillaja extract, gum Arabic, beta lactoglobulin, sodium dodecyl sulfate, soy lecithin, sodium caesinate, potato protein isolate (for example Solanic 300, Avebe®), whey protein isolate, starch octenyl succinate or a combination thereof. Preferably from polyvinylalcohol, a polysorbate, such as Tween 20 or Tween 80, beta lactoglobulin and starch octenyl succinate. Polyvinylalcohol provides an excellent monodispersity of the droplets formed. Furthermore, the first surfactant may be a solid particle, depending on the application preferably a hydrophobic hydrophilic or Janus-type particle, configured for providing a pickering emulsion. For example, the solid particle may be colloidal silica. If PVA shall be avoided, gum arabic, Tween 20, potato protein, pectin or mixtures thereof may be employed as first surfactant, respectively as PVA replacement. Furthermore, the first surfactant may be a solid particle, depending on the application preferably a hydrophobic hydrophilic or Janus-type particle, configured for providing a pickering emulsion. For example, the solid particle may be colloidal silica.

In some embodiments, the amount of the at least one surfactant, respectively all employed first surfactants, in the continuous aqueous phase is in the range of 0.5 wt % to 5 wt %, in particular 0.5 wt % to 2 wt %.

As used herein, the term “microcapsule” generally refers to a capsule with a particle size of less than 4 mm, preferably between 1 μm and <4 mm, more preferably between 1 μm and <1 mm, more preferably between 10 μm and <1 mm. Concomitantly, a microdroplet has a droplet size, i.e. a diameter of less than 4 mm, preferably between 1 μm and <4 mm, more preferably between 1 μm and <1 mm and a microchannel has a diameter of typically less than 4 mm, preferably between 1 μm and <4 mm, more preferably between 25 μm and <3.5 mm, more preferably between 25 μm and <1.3 mm. The term “capsule” as used herein does not only comprise capsules having a capsule wall fully enclosing a void, but also massive capsules, such as beads.

Steps a. to c. may in particular be performed in an emulsification device as it is disclosed in WO 2021 037 999 A2 of the applicant, which is included herein by reference in its entirety, in particular an emulsification device as it is disclosed in the independent claim, the dependent claims as well as FIG. 1-3 and the corresponding figure description. Preferably, the emulsification device may be a device for generating a dispersion of a first phase (i.e. the droplet phase) in a second phase (i.e. the continuous aqueous phase), the emulsification device comprising a first inlet for supplying a first phase, which opens into a first chamber, a second inlet for supplying a second phase, opening into a second chamber and a dispersion outlet for collecting the dispersion. Furthermore, the emulsification device comprises a membrane, which separates the first chamber and the second chamber and which comprises a first side facing the first chamber and a second side facing the second chamber. The membrane comprises multiple channels extending from the first side to the second side, providing a fluidic connection between the first chamber and the second chamber. Each channel comprises a channel inlet arranged on the first side and a channel outlet arranged on the second side. The first chamber may typically be configured such that a flow rate of the first phase through all of the individual channels is essentially equal. Such an emulsification device may for example be fluidic connected via its dispersion outlet to the first fluid inlet of the bottom portion of the gelation device.

The first chamber and second chamber are typically separated from each other with the exception of the one or more channels connecting the first chamber with the second chamber. A chamber as used herein is configured for being filled with a solution. Typically, the chambers are hermetically closed with the exception of inlets, channels and outlets. However, the chambers, in particular the second chamber, may also, at least indirectly or directly, be open to the environment.

The first chamber has typically a first fluid inlet for introducing, particularly continuously introducing, the droplet phase in step a. into the first chamber and the second chamber has a second inlet for introducing, particularly continuously introducing, the continuous aqueous phase into the second chamber in step b. The second chamber also has a dispersion outlet for removing, preferably continuously removing, the emulsion or dispersion formed during step c. from the second chamber.

It is understood that the one or more channels each comprise an inlet opening into the first chamber and an outlet opening into the second chamber. Thus, the one or more channels are directly connected to the first chamber and the second chamber. Typically, the first chamber and the second chamber are fluidic connected by multiple channels, i.e. at least 10, at least 20, at least 30, at least 50 or at least 100 channels. Preferably, the first chamber and the second chamber are fluidic connected by 1 to 30 000 000, preferably 20 to 1 000 000,more preferably 100 to 1 000 000 channels. Typically, the channels are arranged essentially parallel to each other.

For example, the one or more channels may have a diameter in the range of 0.25 μm to 2000 μm, preferably 2 μm to 1200 μm, in particular of 5 μm to 1100 μm. The channels are typically micro-channels. For example, each channel may have a cross-sectional area of 0.04 μm² to 4 000 000 μm², preferably 4 μm² to 640 000 μm².

In some embodiments channel length is in the range of 0.05 mm to 20 mm, particularly between 0.1 mm to 20 mm, particularly 0.1 mm to 5 mm, particularly 0.5 to 20 mm.

In further embodiments, the aspect ratio of each channel, which is defined as channel length/minimum diameter, is in the range of 5 to 1000, particularly, 10 to 500, more particularly 10 to 50. In some embodiments, the length of the channel may be in the range of 0.05 mm to 20 mm, particularly between 0.1 mm to 20 mm, particularly 0.1 mm to 5 mm, particularly 0.5 to 20 mm.

In certain embodiments each channel comprises a channel outlet with a cross-sectional area which is larger than the cross-sectional area of the remaining part of the respective channel. In the longitudinal direction, i.e. in the direction of flow, the channel outlet has a typical length of several micrometers, for example 200 μm to 20 mm, preferably 500 μm to 5 mm. The channel outlet may for example be funnel shaped, V-shaped or U-shaped. In some embodiments, the channel outlet may have an elliptical contour. In particular, the channel outlet is not rotational symmetric, thus having a ratio of length/width of 3 and higher. Hence, the channel outlet may not have a circular or square shaped cross-section. Such a channel outlet enables the detachment of a droplet without external force. As a result, droplet formation of the droplet phase in the continuous aqueous phase is decoupled and thus essentially independent from the flow rate. According to the Young-Laplace equation, the pressure at an immiscible liquid interface is higher at the channel outlets than in the second chamber. Thus a pressure gradient along the direction of the flow is generated, which causes the detachment of the fluid thread into individual droplets. Thus a pressure gradient is generated at the end of the channel, which facilitates the detachment of the fluids boundary layer and therefore the formation of the individual droplets. When reaching the channel outlet, the pressure gradient of the droplet phase in and outside of the channel a droplet detaches without external force. Such a nozzle is advantageous, as it decouples the flow rates from the emulsification process.

Typically, each channel is defined by channel walls. The channel walls may be curved, i.e. the channel walls may be convexly or concavely shaped towards the channel outlet. Furthermore, each channel may comprise a constriction with a cross-section which is smaller than the cross-section of the rest of the channel and wherein the constriction is arranged adjacent the channel outlet. Thus, the constriction is arranged between the channel outlet and the rest of the channel.

In further embodiments, the cross-sectional area of each channel outlet is 0.12 to 36 000 000 μm², preferably 12 to 5 760 000 μm². In particular, in embodiments in which the channels are comprised in a membrane, total open area of the second side of the membrane may be 300% to 1500%, preferably 400% to 900%, larger than total open area of the channels at any other given position, such as the main section and/or the channel inlets.

In some embodiments, the one or more channels may be comprised in a membrane separating the first chamber from the second chamber. In such embodiments, the membrane can be flat, for example disc-shaped. The membrane typically has a first side facing the first chamber and a second side, being opposite to the first side and facing the second chamber. Thus, the first side of the membrane may partially limit and/or define the first chamber and the second side of the membrane may partially limit and/or define the second chamber. The one or more channels, typically multiple channels, extend from the first side to the second side through the membrane. Each channel comprises a channel inlet arranged at the first side, a channel outlet arranged at the second side and a main section being arranged between the channel inlet and channel outlet, wherein the channel outlet comprises a shape deviating from the shape of the main section.

In some embodiments the membrane thickness is in the range of 0.05 mm to 25 mm, particularly between 0.1 mm to 25 mm, particularly 0.5 to 25 mm particularly 0.1 mm to 5 mm. Typically, the thickness of the membrane may be equal to the total length of each channel.

The membrane may typically be a monolayer membrane. That is, the membrane is made from a single piece. Preferably, such a membrane is made from a massive material and does not contain any phase interfaces or transition areas in addition to the multiple channels of the membrane. Such a membrane is advantageous for the quality of the generated droplets, as any phase interfaces and transitions are detrimental to droplet formation and droplet stability.

In some embodiments, the membrane may be exchangeable. The multiple channels of the membrane are typically micro-channels. For example, each channel may have a cross-sectional area in the range of 0.04 μm² to 4 000 000 μm², preferably 4 μm² to 640 000 μm².

In further embodiments, the channel outlet may be wedge-shaped. In particular, the channel outlet may comprise an elliptical cross-section with respect to a transversal plane being perpendicular to the extending channel, i.e. the channel outlet may be larger in a first direction than in a second direction.

In further embodiments, the second side of the membrane comprises a total open area that is larger than the total open area of the first side. Such a membrane has the advantage that high quality droplets are generated, even at flow rates of up to 5 L/h. In some embodiments, the flow rate per channel may be in the range of 1 μL/h to 50 mL/h, preferably 10 μL/h to 5 mL/h.

In certain embodiments, each channel outlet may have an elliptical contour. Thus, the channel outlet may have an elliptical cross-section with respect to a plane being transversal to the extending channel and being parallel to the first or second side of the membrane. Channel outlets with an elliptical contour have a beneficial effect on the quality of the formed droplets, as any edges within the channel may lead to unstable and inhomogeneous droplets.

In some embodiments, the membrane is disk-shaped. Such a membrane may have a circular contour. Alternatively, the membrane may have an angular, particularly a triangular or rectangular, contour.

In further embodiments, the membrane comprises 0.06 to 600 000 channels/cm², preferably 20 to 30 000 channels/cm².

In some embodiments, the droplet phase is provided into the first chamber with a pressure of 5 mbar to 300 mbar, in particular 20 mbar to 100 mbar, above atmospheric pressure.

In some embodiments, the continuous aqueous phase is provided into the second chamber with a pressure of 20 mbar to 200 mbar, in particular 50 mbar to 100 mbar, above atmospheric pressure.

In some embodiments, the first operating temperature is in the range of 20° C. and 100° C., in particular in the range of 20° C. and 95° C., in particular in the range of 40° C. and 75° C.

The first operating temperature may be provided by heating the droplet phase to the first operating temperature before it is provided in the first chamber and/or via heating elements being configured such that they heat the droplet phase in the first chamber to the first operating temperature.

In some embodiments the second operating temperature is in the range of 20° C. and 100° C., in particular in the range of 20° C. and 95° C., in particular in the range of 40° C. and 75° C. The second operating temperature may be provided by heating the droplet phase to the second operating temperature before it is provided in the first chamber and/or via heating elements being configured such that they heat the droplet phase in the first chamber to the second operating temperature.

In some embodiments, the storage temperature is 40° C. or less, in particular in the range of −80° C. and 40° C., in particular in the range of −15° C. and 40° C., in particular in the range of 0° C. and 35° C., in particular in the range of 10° C. and 35° C.

Using a hydrophobic matrix with such first and/or second operating temperatures and such a storage temperature has the advantage that microcapsules can be generated and optionally loaded with an active ingredient in an efficient manner. At first, due to the relatively small temperature difference, the overall process does not require high amounts of energy. Furthermore, using a hydrophobic matrix which is liquid at a first operating temperature of only in the range of 20° C. and 100° C., in particular in the range of 20° C. and 95° C., in particular in the range of 40° C. and 75° C., has the advantage that the capsules are particularly suitable for example for food industry applications, as the capsules may be molten during cooking or for pharmaceutical applications as the capsules may melt when exposed to normal human body temperatures after intake.

In some embodiments, the first operating temperature is the same or higher as the second operating temperature. The first operating temperature and/or the second operating temperature may be provided by heating elements of the first chamber and/or the second chamber. Additionally, or alternatively, the first operating temperature and/or the second operating temperature may be provided by pre-heating or pre-cooling of the droplet phase and/or the continuous aqueous phase before being provided into the first, respectively second, chamber.

In some embodiments the second temperature is equal or higher than the storage temperature. Typically, the second temperature is selected such that the hydrophobic matrix is still liquid or that it only partially but not fully solidifies. It may however also be possible to select the second temperature such that the hydrophobic matrix solidifies, e.g. in the second chamber.

In some embodiments, the droplet phase in step a. additionally comprises at least one compound of interest. The compound of interest may be selected from a protein, small molecule, particularly a fragrant or flavor, a coloring agent, cosmetic ingredient, active pharmaceutical ingredient such as cannabinoids, hemp extracts, caffeine, melatonin or hyaluronic acid; antibody, peptide, enzyme, RNA, DNA, vitamin and micro-organisms, including bacteria, phages, algae, cells, fungi, and bacteria supporting components, such as sugars, buffers, nutrients, reduction and oxidizing agents, etc. The at least one compound of interest may for example be mixed into the droplet phase in a suitable concentration. As the compound of interest can be homogenously mixed and distributed within the droplet phase, the compound is then also homogenously distributed within the generated capsules.

In particular embodiments, the at least one compound of interest being comprised in the droplet phase of step a. is hydrophobic. Typically, the at least one compound of interest is soluble in the liquid hydrophobic matrix at ambient temperature (20-25° C.) and pressure (1 atm.).

In some embodiments, step a. comprises liquefying the hydrophobic matrix to form the liquid hydrophobic matrix. The at least one compound of interest may already be mixed or dissolved in the liquid hydrophobic matrix, it may be added after the liquefaction of the hydrophobic matrix or it may be added during liquefaction of the hydrophobic matrix.

In specific embodiments, the at least one compound of interest is a living organism, in particular a microorganism, such as bacteria, a virus, including a phage, or a single cell. In some embodiments, the living organism may be provided in a dormant state into the droplet phase. It is understood that the dormant state of a living organism relates to an inactive state.

The method according to the invention is particularly suitable for encapsulating living organisms, because the method exerts only marginal shear forces as compared to the method of the prior art. Furthermore, the encapsulation efficiency is significantly higher than of methods known in the prior art. It is possible to reach encapsulation efficiencies of up to 90% or even up to 95% with respect to the living organism.

In some embodiments, the method is performed at room temperature, which is highly beneficial for encapsulating living organisms, as the viability is increased.

Furthermore, by guiding the droplet phase through the one or more channels, the channel dimension, in particular the channel diameter, dictates the amounts of living organism per droplet and thus the mount of organism per capsule formed. Therefore, by choosing predefined channel dimensions, accurate control of organism loading per capsule is possible.

In some embodiments, the channel diameter is chosen such that it is at least 6 times larger than the size of the living organism, or also any other solid particle.

In some embodiments in which the at least one compound of interest is a living organism, the living organism, such as cells or bacteria, is provided by cultivation prior to being added into the droplet phase. For example, cultivation may be performed on a suitable nutrient medium, such as agar-agar. In certain embodiments, viability of the living organism is monitored during cultivation and the living organism freeze dried when the viability reaches its maximum and subsequently added to the droplet phase.

In certain embodiments it is beneficial to deoxygenate the droplet phase and/or the continuous aqueous phase. Deoxygenating can be achieved by common laboratory techniques, such as degassing with inert gases, such as argon or nitrogen, or by the freeze-pump-thaw technique. Such deoxygenating is beneficial, because the living organism can be maintained in its dormant state.

In some embodiments, the droplet phase additionally comprises nutritional components for the living microorganism, such as sugars, electrolyte solutions, and the like.

In some embodiments, the at least one compound of interest is hydrophobic and the concentration of the compound of interest in the droplet phase is up to 50 wt %, in particular up to 35 wt %.

In some embodiments, the at least one compound of interest is a powder being dispersed in the droplet phase. Thus, in such embodiments, the droplet phase may be a dispersion, respectively a suspension, in particular a micro-dispersion, respectively micro-suspension. In certain embodiments, the concentration of the powder in the droplet phase is up to 50 wt %, in particular up to 40 wt %, in particular up to 30 wt %. In some embodiments, the channel diameter is chosen such that it is at least 6 times larger than the size of the powder particle. The particle size of such a powder particle or any other particle may for example be determined by laser diffraction or static light scattering.

In some embodiments, the droplet phase provided in the first chamber in step a. is an emulsion of an aqueous droplet phase in the liquid hydrophobic matrix. The droplet phase further comprises at least one second surfactant. Thus in such embodiments, the droplet phase of step a. is an emulsion of an aqueous droplet phase in the liquid hydrophobic matrix (i.e. a water-in-oil emulsion). The second surfactant being additionally present in the droplet phase in such embodiments stabilizes this emulsion. Such embodiments are particularly suitable if the compound of interest is hydrophilic and thus not soluble or only hardly soluble in the liquid hydrophobic matrix. This water-in-oil emulsion is then, after it has been provided to the first chamber, guided through the one or more channels in the second chamber upon which it forms a water-in-oil-in-water emulsion or dispersion. In some embodiments, the maximum amount of the aqueous droplet phase in the droplet phase is 40 wt %. Thus, the amount of the hydrophobic matrix in the droplet phase is at least 60 wt %. It is further possible that the aqueous droplet phase consists of the at least one compound of interest, that is, the aqueous droplet phase is the compound of interest. This is for example possible if the compound of interest is liquid at the first operating temperature. For example, the compound of interest may be a water soluble amino acid being liquid at the first operating temperature. In such embodiments, it may be possible to directly use the compound of interest as the aqueous droplet phase. It is therefore clear to the skilled person that the term “aqueous droplet phase” does not necessarily mean that the aqueous droplet phase must comprise water, however it means that the aqueous droplet phase is miscible with water and immiscible with the liquid hydrophobic matrix.

It has been observed that such embodiments, i.e. embodiments which employ a water-in-oil-in-water emulsion are highly effective for encapsulating hydrophilic compounds of interest in a hydrophobic matrix. For example, by using caffeine as a hydrophilic compound of interest, it was possible to encapsulate at least 90 wt % to 95 wt % of the hydrophilic compound initially provided in the droplet phase of step a.

In some embodiments, the second surfactant may be a nonionic surfactant, such as polyglycerol polyricinoleate (PGPR), polyvinyl alcohol (PVA) or Span derivatives, such as Span 80 or Span 85 or a combination thereof. Furthermore, the second surfactant may be a solid particle, depending on the application preferably a hydrophobic hydrophilic or Janus-type particle, configured for providing a pickering emulsion. For example, the solid particle may be colloidal silica.

In certain embodiments, the droplet phase is therefore an emulsion of an aqueous droplet phase in the liquid hydrophobic matrix as described above and the aqueous droplet phase comprises a hydrophilic compound of interest. In some embodiments the concentration of the hydrophilic compound of interest in the droplet phase is 10 wt % to 15 wt %.

In some embodiments, step d. is performed in a cooling bath in a batch reactor, in particular an aqueous cooling bath. For example, after step c. the formed emulsion or dispersion of the droplet phase in the continuous aqueous phase is guided into a batch reactor containing water at a temperature which is lower than the first and/or second operating temperature. This temperature of the water may be considered as the cooling temperature. In particular, the water in the batch reactor may be equal or below the storage temperature or may be equal or below the transition temperature. The cooling temperature is preferably at least 10° C., in particular at least 12° C. below the transition temperature of the hydrophobic matrix. Particularly, the cooling temperature is in the range of 10° C. and 20° C. below the transition temperature of the hydrophobic matrix. Such temperatures are advantageous, since particularly a pure hydrophobic matrix material without small impurities which typically accelerate solidification tend to only slowly solidify if a higher temperature is selected. Furthermore, the stable and reliable capsules are produced.

In certain embodiments, the batch reactor may be equipped with a stirrer. The formed emulsion or dispersion of the droplet phase in the continuous aqueous phase is then stirred within the batch reactor by the stirrer.

Alternatively, in some embodiments step d. is performed in a cooling column by guiding the formed emulsion or dispersion of the droplet phase in the continuous aqueous phase through the cooling column. While guiding the formed emulsion or dispersion of the droplet phase in the continuous aqueous phase through the cooling column, the cooling column cools the formed emulsion or dispersion of the droplet phase in the continuous aqueous phase. Using such a cooling column makes the overall production much more efficient, in particular as a continuous process can be used and not a batch-wise process. Furthermore, such a continuous process ensures that all capsules formed are exposed to the same cooling conditions, i.e. are exposed to the cooling for the same time period, which increases the capsule homogeneity. The temperature inside the column may be a cooling temperature. Also in such embodiments, the cooling temperature is preferably at least 10° C., in particular at least 12° C. below the transition temperature of the hydrophobic matrix. Particularly, the cooling temperature is in the range of 10° C. and 20° C. below the transition temperature of the hydrophobic matrix. Such temperatures are advantageous, since particularly a pure hydrophobic matrix material without small impurities which typically accelerate solidification tend to only slowly solidify if a higher temperature is selected. Furthermore, the stable and reliable capsules are produced.

In some embodiments, the cooling column comprises one or more cooling elements being configured for cooling the emulsion or dispersion of the droplet phase in the continuous aqueous phase. For example, the cooling elements may be electric cooling elements or a suitable cooling medium such as a cooling liquid or cooling gas.

In some embodiments, the formed emulsion or dispersion of the droplet phase in the continuous aqueous phase is guided through the cooling column such that is fluidic separated from a cooling medium. For example, a condenser may be used as the cooling column, which has an inner flow path for the formed emulsion or dispersion of the droplet phase in the continuous aqueous phase which is circumferentially surrounded by an outer flow path for the cooling fluid or the cooling gas.

In some embodiments, the formed emulsion or dispersion of the droplet phase in the continuous aqueous phase is guided through the cooling column such that is mixed with a cooling medium. Thus, the cooling medium and the formed emulsion or dispersion of the droplet phase in the continuous aqueous phase physically contact each other. In particular, the cooling medium may be water. In certain embodiments, the cooling medium may the same, respectively have the same composition than the continuous aqueous phase provided in step a.

In certain embodiments, the cooling column may comprise a tubular column, which has a longitudinal axis which extends along an axial direction of the tubular column. Typically, the axial direction is perpendicular to the radial direction of the tubular column. The cooling column further comprises a bottom portion and a head portion. Typically, the tubular column is arranged between the bottom portion and the head portion. The bottom portion comprises a first fluid inlet which is configured for introducing a droplet phase into the tubular column.

Additionally, the cooling column and in particular the bottom portion, comprises another, second fluid inlet, which is configured for introducing a continuous aqueous phase into the tubular column. Such a continuous aqueous phase may be used as cooling medium, i.e. it may have a temperature below the transition temperature of the liquid hydrophobic matrix or it may have storage temperature. This continuous aqueous phase may consist of water or it may have the same composition as the continuous aqueous phase of step b. of the method according to the invention. The head portion comprises a fluid outlet which is configured for removing capsules with the solid matrix from the tubular column. The capsules with the solid matrix are typically dispersed in the continuous aqueous phase which is introduced into the tubular column via the second fluid inlet. The cooling column further comprises a stirring device which is arranged inside the tubular column. The stirring device comprises one or more stirring elements, which are each longitudinally arranged inside the tubular column and which are each rotatable around the longitudinal axis of the tubular column. Each stirring element is configured to provide for a radial mixing of the droplet phase and the continuous aqueous phase, i.e. the droplet phase being introducible via the first fluid inlet and the continuous aqueous phase being introducible via the second fluid inlet. Optionally, each stirring element is configured to essentially avoid axial mixing of the droplet phase and the continuous aqueous phase.

It is understood that the longitudinal axis of the tubular column is arranged in the center of the tubular column. In particular, the longitudinal axis has in any radial direction the same distance to the column walls.

Longitudinally arranged stirring elements are elongated along the longitudinal axis, i.e. in the axial direction of the tubular column, that is, in contrast to a radially arranged element which would be elongated along the radial direction. Typically, the extension of each stirring element in the axial direction of the tubular column is larger than the extension in the radial direction of the tubular column, particularly at least 10-fold, e.g. between 20-to 30-fold, larger. In some embodiments, each stirring element may extend essentially in parallel to the longitudinal axis of the tubular column. “Essentially in parallel” includes also stirring elements which are inclined by 10° or less, in particular 5° or less, with respect to the longitudinal axis of the tubular column. Preferably, the stirring elements are arranged in parallel to the longitudinal axis. Typically, the longitudinally arranged stirring elements extend in the axial direction of the tubular column.

Each stirring element may extend through the tubular column, in particular in the axial direction through at least 50%, particularly through at least 75%, particularly through at least 85%, particularly through at least 90%, particularly completely through, the tubular column.

By employing one or more stirring elements as described, the droplet phase comprising droplets and/or capsules, in particular growing or solidifying capsules, are constantly kept in motion, which prevents agglomeration as well as aggregation. Furthermore, as the droplet phase is continuously guided through the column in its axial direction in presence of the continuous aqueous phase without axial mixing, the residence time distribution over all capsules is narrow, which ensures a uniform capsule size and quality. Additionally, employing longitudinally arranged stirring elements allows for providing a constant mixing over the column, which ensures uniformity of the capsules and also helps to prevent blockage of the column.

The tubular column is in some embodiments cylindrical. In particular, the tubular column may define a cylindrical chamber. It is understood that the tubular column has a head opening and a bottom opening. Typically, the bottom portion of the cooling column is attached to the bottom opening of the tubular column and the head portion of the cooling is attached to the head opening of the tubular column. It is understood that the terms “top” and “bottom” do not necessarily mean that the top portion is in the 3D space in the vertical direction (i.e. against the gravitational force vector) necessarily above the bottom portion. Furthermore, it may be possible to use the device in a horizontal manner, i.e. 90° to the gravitational force vector. The tubular column has column walls, which define the column chamber, i.e. the chamber in which the stirring device is arranged. In some embodiments, the tubular column may essentially be rotationally symmetric with respect to its longitudinal axis.

In certain embodiments, each stirring element is configured such that it has a rotation path, in particular a circular rotation path or an epicycloid rotation path, when it rotates around the longitudinal axis of the tubular column. In specific embodiments, each rotation path of each stirring element is concentric with the column walls.

In some embodiments, the length, i.e. the extension of the tubular column in the axial direction, to width, respectively the diameter, of the tubular column is at least 5:1, in particular at least 10:1. Particularly, the length to width of the tubular column is in the range of 5:1 and 40:1, in particular in the range of 10:1 and 20:1. Thus, the flow behavior within the tubular column typically resembles a pipe flow. In particular, the length of the tubular column may in some embodiments be in the range of 5 cm and 200 cm, in particular in the range of 20 cm and 150 cm, in particular in the range of 50 cm and 100 cm. In some embodiments, the width, respectively the diameter of the tubular column is in the range of 5 mm and 200 mm, in particular in the range of 20 mm and 150 mm, in particular in the range of 50 mm and 100 mm.

In some embodiments, the stirring device, and in particular the one or more stirring elements are configured such that a radial vortex is generated when mixing the droplet phase and the continuous aqueous phase.

In some embodiments, the stirring device comprises in the range of 1 and 24, in particular in the range of 2 and 24, in particular in the range of 3 and 18, in particular in the range of 6 and 12, stirring elements.

In some embodiments, the stirring elements are rods, in particular straight rods. For example, the stirring elements may be cylindrical rods. It is also possible that the rods have a helical shape, i.e. the extend along the axial direction in a helical manner. In other embodiments, the stirring elements are tubes or plates.

In some embodiments, the rods may have a diameter of 1 mm to 30 mm, in particular of 2 mm to 10 mm, in particular of 4 mm to 8 mm.

In some embodiments, the one or more stirring elements are free of radial surfaces. A radial surface is a surface which is arranged such that a particle being guided in the axial direction from the first or second fluid inlet to the fluid outlet through the tubular column can be retained or blocked. Such embodiments are advantageous, because any agglomeration under such surfaces is avoided, which prevents blocking and reduced capsule quality, in particular reduced homogeneity.

In some embodiments, the stirring device comprises one or more groups of stirring elements. Each of these groups comprises at least two stirring elements being arranged around a common group axis, which is arranged essentially in parallel to the longitudinal axis of the tubular column. As mentioned above, the term “essentially in parallel” includes also group axes which are inclined by 10° or less, in particular 5° or less, with respect to the longitudinal axis of the tubular column. Each stirring element of each group is in these embodiments additionally rotatable around the common group axis. Thus, each stirring element can undergo two rotations, namely a rotation around the longitudinal axis of the tubular column and additionally a rotation along the corresponding common group axis. In other words, the stirring elements are not only movable in the tangential direction, but also in the radial direction. Such embodiments provide for a profoundly enhanced radial mixing and thus further improve capsule homogeneity.

In some embodiments, the cooling column comprises a tubular column, which defines, respectively comprises, a longitudinally arranged dispersion channel. Such a longitudinally arranged dispersion channel is a channel which extends in the longitudinal direction of the tubular column and whose extension in the longitudinal direction, i.e. its length, is larger than its extension in the radial direction, i.e. its width, respectively diameter. The emulsion or dispersion of the droplet phase in the continuous aqueous phase is transported through the dispersion channel along the longitudinal direction of the tubular column through the tubular column. Thus, it is understood, that the dispersion channel typically has a dispersion channel inlet through which the emulsion or dispersion of the droplet phase in the continuous aqueous phase is introduced and a dispersion channel outlet through which the capsules with the solid matrix are removed. The tubular column further comprises a first mesh unit. The first mesh unit may typically comprise a filter element. Furthermore, the cooling column comprises a cross-flow fluid inlet unit. The cross-flow fluid inlet unit introduces a cross-flow fluid into the dispersion channel in such a way that the introduced cross-flow fluid flows transversely, and in particular perpendicularly, to the longitudinal direction of the tubular column. Thus, the cross-flow fluid inlet unit introduces a cross-flow fluid into the dispersion channel such that the introduced cross-flow fluid flows transversely, particularly perpendicularly, to the emulsion or dispersion of the droplet phase in the continuous aqueous phase being transported through the tubular column. The cross-flow fluid has a temperature which is lower than the first and/or second operating temperature and in particular which is lower than the transition temperature or the storage temperature of the hydrophobic matrix.

Furthermore, the cross-flow fluid inlet unit is configured such that the cross flow fluid flows through the first mesh unit. This has the advantage that the cross-flow fluid may replace and/or remove the continuous aqueous phase of the emulsion or dispersion which is introduced into the dispersion channel. The first mesh unit thereby avoids that the solidifying capsules are removed from the dispersion channel. Thus, the first mesh unit typically has a mesh with a mesh size which is smaller than the diameter of the capsules having a solid matrix. The advantage of providing a cross-flow fluid in this manner is that the cross-flow fluid flows radially, while the generated capsules flow longitudinally through the dispersion channel. Furthermore, the cooling time of each capsule is equal to the cooling time of all other capsules, which increases the homogeneity of the capsules.

The cross-flow fluid may in particular be water, or it may have the same composition as the continuous aqueous phase provided in step b. of the method according to the invention.

It is understood that the cross-flow fluid inlet unit typically comprises an inlet port through which the cross-flow fluid can be introduced into the cross-flow fluid inlet unit and an outlet port, such as multiple openings, through which the cross-flow fluid can be expelled from the cross-flow fluid inlet unit into the dispersion channel.

In some embodiments, the cross-flow fluid inlet unit comprises a second mesh unit which introduces the cross-flow fluid into the dispersion channel. It is understood that the first and second mesh unit are different and typically separate from each other. In certain embodiments, the second mesh unit introduces the cross-flow fluid transversely, in particular perpendicularly, to the longitudinal direction of the tubular column, respectively the dispersion channel. In other words, the second mesh unit introduces the cross-flow fluid radially into the dispersion channel. Typically, the second mesh unit is arranged inside the tubular column.

The second mesh unit and the first mesh unit are typically spaced apart from each other such that they define the dispersion channel between them. The dispersion channel, the first mesh unit and the second mesh unit are typically coaxially arranged to each other.

In some embodiments, the second mesh unit and the first mesh unit are aligned with each other, i.e. fluid being introduced radially from the second mesh unit into the dispersion channel is directly guided, i.e. linearly and radially, towards the first mesh unit.

In some embodiments, the tubular column, the first mesh unit, the second mesh unit have the shape of a cylinder. Furthermore, also the dispersion channel may have the shape of a cylinder.

In some embodiments, the hydrophobic matrix comprises or consists of a wax, a fat and/or an oil, in particular a hydrogenated oil or a vegetarian oil. Suitable vegetarian oils are rapeseed oil, palm kernel oil, sunflower seed oil, hemp oil, canola oil, palm oil, soybean oil, coconut oil, olive oil and the like.

In some embodiments, the hydrophobic matrix is configured such that it has a melting point at which 100% of the hydrophobic matrix is liquid in the range of 20° C. to 90° C., in particular in the range of 40° C. to 75° C.

In some embodiments, the hydrophobic matrix is configured such that it has a melting starting point in the range of 20° C. to 90° C., in particular in the range of 40° C. to 75° C. . . . The melting starting point is the temperature at which the hydrophobic matrix starts to become liquid.

In some embodiments, the hydrophobic matrix is selected such that it has a melting range interval in the range of 5 K to 45 K, in particular in the range of 5 K to 20 K. The melting range interval is the temperature range between the melting starting point and the melting point at which 100% of the hydrophobic matrix is liquid.

In some embodiments, a mixture of a plurality of different hydrophilic matrixes is used in step a. For example, a mixture of a hydrogenated oil and for example another oil, such as coconut oil, is used in combination. In certain embodiments, the plurality of different hydrophilic matrixes is mixed in such a ratio that the resulting mixture has a melting starting point in the range of 15° C. to 85° C., in particular in the range of 36° C. to 70° C., and/or a melting point at which 100% of the hydrophobic matrix mixture is liquid in the range of 20° C. to 90° C., in particular in the range of 40° C. to 75° C., and/or a melting range interval in the range of 5 K to 45 K, in particular in the range of 5 K to 20 K.

In some embodiments, the capsules with a solid matrix are dried after step d. In certain embodiments, the capsules are first filtered to remove the majority of the continuous aqueous phase. Drying may comprise heating the capsule to a drying temperature. Importantly, the drying temperature is selected such that it is lower than the first temperature and/or the transition temperature in order to avoid melting or necking (capsules starting to fuse together) of the capsules. Alternatively, or additionally, drying may be achieved by applying sub-atmospheric pressure, in particular high vacuum of below 1 mbar, in particular at 25° C. or even below or medium vacuum, i.e. between 1 mbar and 900 mbar, in particular 1 mbar and 600 mbar, and a temperature of above 25° C.

A second aspect of the invention relates to an assembly of capsules, particularly microcapsules, comprising a plurality of capsules produced according to the method as described in any of the embodiments herein, in particular with respect to the first aspect of the invention.

In some embodiments of the assembly of capsules the capsules have an equal size distribution with a coefficient of variation of 10% or less, particularly of 8% or less, particularly of 6% or less, particularly of 5% or less, particularly of 4% or less.

The skilled person understands that the coefficient of variation may be calculated by the ratio of the standard deviation o to the mean u, i.e. the average capsule size of the capsules of the assembly.

In some embodiments, the assembly of capsules comprises more than 50 capsules, particularly more than 100 capsules, particularly more than 500 capsules, particularly more than 1000 capsules, particularly more than 10 000 capsules, produced according to the method according to any of the embodiments described herein.

In some embodiments, each capsule of the assembly of capsules has a particle size of less than 4 mm, preferably in the range of 1 μm and <4 mm, more preferably in the range of 1 μm and <1 mm.

In some embodiments, the capsules of the assembly, in particular all capsules of the assembly, have a maximum difference of 1% with respect to a perfect sphere. In particular, the surface of the capsules has a maximum difference of 5% or even of maximum 1% with respect to a perfect sphere.

In some embodiments, the capsules of the assembly comprise a solid matrix which melts at a temperature of at least 20° C., in particular of at least 40° C. Such embodiments, have the advantage that a compound of interest within the capsules is released at a specific, predetermined temperature.

Capsules produced by the method according to the invention and thus, also an assembly according to any of the embodiments of the second aspect of the invention were able to maintain even volatile compounds within the capsules even at temperatures being higher than ambient temperature (20-25° C.). For example, a volatile compound of interest having been encapsulated into a capsule by the method according to the invention essentially remained inside the capsules even after being maintained at 54° C. for a period of 2 weeks, while a comparative amount of the compound itself (without being encapsulated) completely evaporated under the same conditions.

A third aspect of the invention, relates to an emulsion or dispersion of microdroplets, being particularly obtained via steps a. to c. of the method according to any of the embodiments described herein. The dispersion comprises a continuous aqueous phase and microdroplets of the droplet phase. The dispersion of microdroplets may further comprise a first surfactant.

A fourth aspect of the invention relates to a method for generating a food product. Such a method comprises the method for generating capsules with a solid matrix, respectively its steps, according to any of the embodiments as described herein, in particular with respect to the first aspect of the invention. Furthermore, the method comprises combining the generated capsule/capsules with a solid matrix with ingredients of a food product to generate the food product.

The food product may be any suitable food product, however preferably the food product is a meat substitute food product. Meat substitute food product are also known as “meat analogues”, “plant-based meat”, “vegan meat” and the like. Meat substitute products have a similar or equal protein content than meat of animal origin and may further haptically be similar or equal to meat. For example, meat substitute food products may be soy based or pea based. In some examples the meat substitute food product may be a burger patty.

In some embodiments, the generated capsule comprises at least one compound of interest as described herein, in particular with respect to the first aspect. In certain embodiments, the at least one compound of interest may be a flavor or a coloring agent, in particular a food coloring agent, such as beetroot, particularly beetroot extract or juice. The hydrophobic matrix itself may increase the juiciness of the food product, in particular if the hydrophobic matrix is a suitable oil or fat.

A fifth aspect of the invention relates to a method for generating a cosmetic product. The method comprises the method according to any of the embodiments, respectively its steps, as described herein, in particular with respect to the first aspect and further comprising combining the generated capsule with a solid matrix with one or more cosmetic ingredients of a cosmetic product to generate the cosmetic product.

In some embodiments, the cosmetic product may be a skin care product. In some embodiments, the one or more cosmetic ingredients are selected from a moisturizer, a skin cream, an active agent such as hyaluronic acid, a lotion, a fragrant and the like.

In some embodiments, the generated capsule comprises at least one compound of interest as described herein, in particular with respect to the first aspect and/or the fourth aspect of the invention. In particular, the compound may be an agent having a therapeutic of cosmetic effect, such as hyaluronic acid.

A sixth aspect of the invention relates to the use of an assembly of capsules according to any of the embodiments as described herein, in particular with respect to the second aspect of the invention, in a food product.

In some embodiments, the use may be a use in a food product for food coloring and/or flavor incorporation and/or increasing the juiciness of the food product.

The food product may be a food product as described herein, in particular with respect to the fourth aspect of the invention.

The generated capsule comprises at least one compound of interest as described herein, in particular with respect to the first aspect and/or the fourth aspect of the invention.

A sixth aspect of the invention relates to the use of an assembly of capsules according to any of the embodiments as described herein, in particular with respect to the second aspect of the invention, in a cosmetic product, in particular a skin care product.

The cosmetic product may be a cosmetic product as described herein, in particular with respect to the fifth aspect of the invention.

A seventh aspect of the invention relates to a food product, in particular a meat substitute food product, which comprises the assembly of capsules according to any of the embodiments as described herein, in particular with respect to the second aspect of the invention.

The food product may be a food product as described herein, in particular with respect to the fourth aspect of the invention.

The generated capsule comprises at least one compound of interest as described herein, in particular with respect to the first aspect and/or the fourth aspect of the invention.

An eighth aspect of the invention relates to a cosmetic product, in particular skin care product, comprising the assembly of capsules to any of the embodiments as described herein, in particular with respect to the second aspect of the invention.

The cosmetic product may be a cosmetic product as described herein, in particular with respect to the fifth aspect of the invention.

### BRIEF DESCRIPTION OF THE FIGURES

The herein described invention will be more fully understood from the detailed description given herein below and the accompanying drawings which should not be considered limiting to the invention described in the appended claims. The drawings are showing:

FIG. 1 schematically a method for generating capsules with a solid matrix according to an embodiment of the invention;

FIG. 2 schematically a method for generating capsules with a solid matrix according to another embodiment of the invention;

FIG. 3 schematically a method for generating capsules with a solid matrix according to another embodiment of the invention;

FIG. 4 schematically a method for generating capsules with a solid matrix according to another embodiment of the invention;

FIG. 5 schematically a method for generating capsules with a solid matrix according to another embodiment of the invention;

FIG. 6. a microscopic of a capsule assembly according to an embodiment of the invention with an indicated scale on the bottom right of 0.1 mm;

FIG. 7 an image of a capsule assembly according to another embodiment of the invention with an indicated scale on the bottom right of 1 mm;

FIG. 8 a schematic view of a food product according to an embodiment of the invention;

FIG. 9 a schematic view of a cosmetic product according to an embodiment of the invention.

### EXEMPLARY EMBODIMENTS

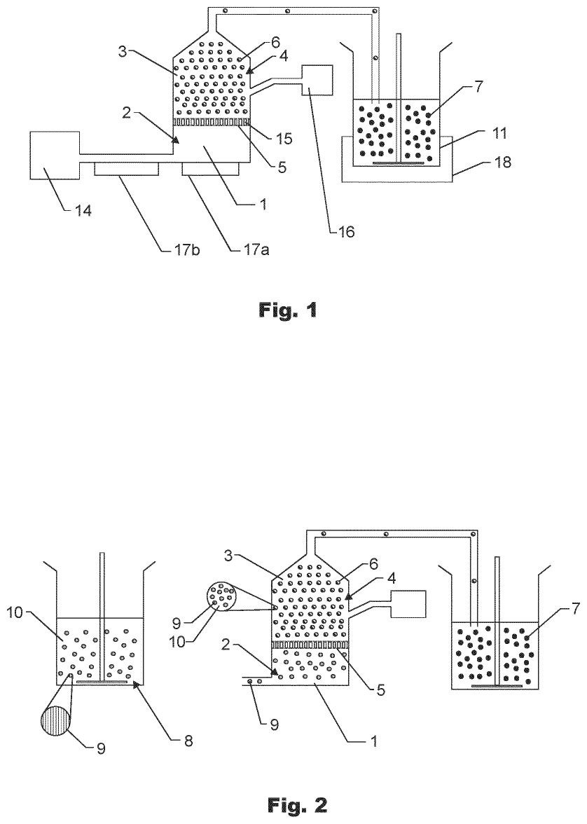

FIG. 1 shows schematically a method for generating capsules with a solid matrix 7. At first, droplet phase 2 is provided from droplet phase reservoir 14 in first chamber 1 at a first operating temperature. Droplet phase 2 comprises a hydrophobic matrix which can be solid at a storage temperature. The first operating temperature is selected such that the hydrophobic matrix is liquid in the first chamber. Furthermore, the first operating temperature is higher than the storage temperature. First chamber 1 may be equipped with heating element 17a which may be an electric heating element, an inductive heating element, a microwave heating element or a thermal fluid heating element and which provides the thermal energy for providing the droplet phase at the first operating temperature. Furthermore, the pipe connecting droplet phase reservoir 14 and first chamber 1 may also be heated by another heating element 17b. In second chamber 3 continuous aqueous phase 4 is provided at a second operating temperature from continuous aqueous phase reservoir 16 via an inlet into the second chamber 3. First chamber 1 and second chamber 3 are fluidic connected with each other by a plurality of channels 5 which are formed in membrane 15 being arranged between first chamber 1 and second chamber 3. Droplet phase 2 is then guided from first chamber 1 through channels 5 into second chamber 3 thereby undergoing a step emulsification and forming an emulsion or dispersion 6 of the droplet phase 2 in continuous aqueous phase 4. Via a dispersion outlet the emulsion or dispersion 6 is then guided into batch reactor 11 being equipped with cooling element 18. The batch reactor comprises a cooling medium, such as a cooling water into which the dispersion or emulsion 6 is guided. Upon contact with the cooling water, the emulsion or dispersion which may have first or second operating temperature and whose hydrophobic matrix is liquid is cooled below the transition temperature of the hydrophobic matrix, which induces a solidification of the hydrophobic matrix thereby forming capsules with solid matrix 7. In order to provide for a homogenous temperature distribution, the cooling water inside batch reactor 11 may be cooled.

FIG. 2 shows another embodiment of the method according to the invention. In this particular case, a hydrophilic compound of interest shall be encapsulated. In order to achieves this, the composition of droplet phase 2 is altered as compared to the embodiment shown in FIG. 1. At first, the hydrophilic compound of interest is dissolved in an aqueous droplet phase 9. This aqueous droplet phase is then added to the liquid hydrophobic matrix 10 in a batch reactor together with a second surfactant. Stirring of the batch reactor produces a water-in-oil emulsion, i.e. aqueous droplet phase 9 being dispersed in liquid hydrophobic matrix 10. It is understood that this mixing may also be done at a temperature being higher than the transition temperature of the hydrophobic matrix in order to ensure that the hydrophobic matrix is liquid. Once the emulsion is formed, this emulsion is used as the droplet phase 2 and is provided in first chamber 1. Then, droplet phase 2 is guided through the plurality of channels 5 into second chamber 3. It is noted that the droplet sizes are exaggerated for clarity purposes and they do therefore not resemble the actual size ratios. Guiding droplet phase 2 through the plurality of channels 5 into second chamber 3, which comprises continuous aqueous phase 4, induces a second emulsification forming emulsion or dispersion 6 of droplet phase 2 in continuous aqueous phase 4. In contrast to the embodiment shown in FIG. 1 however, the formed emulsion or dispersion 6 is now a water-in-oil-in-water emulsion/dispersion. Therefore, every droplet within second chamber 3 consists of microdroplets of aqueous droplet phase 9 being dispersed in liquid hydrophobic matrix 10 (see enlarged view of the corresponding droplet in FIG. 2). Then, the emulsion/dispersion is guided via a dispersion outlet of second chamber 3 into a batch reactor comprising cooling water which is below the transition temperature of the hydrophobic matrix and therefore withdraws thermal energy from emulsion/dispersion 6 until the hydrophobic matrix reaches its transition temperature at which it undergoes a phase transition from the liquid state to the solid state, thereby forming capsules 7 having a solid matrix.

FIG. 3 shows schematically another embodiment of the method according to the invention. Emulsion or dispersion 6 of droplet phase 2 in continuous aqueous phase 4 is generated in a similar manner as in the embodiment of FIG. 1. However, step d., i.e. the cooling step is not performed in a batch reactor as it is the case for the embodiment shown in FIG. 1, but in a continuous manner using cooling column 12. The emulsion or dispersion 6 formed inside second chamber 3 is provided via a dispersion outlet into cooling column 12 via a first fluid inlet of bottom portion 20 of cooling column 12. Additionally, a continuous aqueous phase is introduced from a reservoir 13 via a second fluid inlet into cooling column 12. This continuous aqueous phase is then physically mixed inside cooling column 12 with emulsion/dispersion 6. As the continuous aqueous phase from reservoir 13 is cooler than emulsion/dispersion 6 and typically below the transition temperature of the hydrophobic matrix at which the hydrophobic matrix changes its state of aggregation from liquid to solid, thermal energy is withdrawn by this continuous aqueous phase from the emulsion/dispersion 6 which entails solidification and thus to the formation of capsules 7 having a solid matrix. Cooling column 12 further comprises head portion 19 with a fluid outlet. Furthermore, cooling column 12 comprises stirring device 21 which comprises a plurality of stirring rods as stirring elements. The stirring elements are each longitudinally arranged (i.e. along longitudinal direction LO) inside the tubular column which is arranged between the head portion and the bottom portion of cooling column 12 and which are each rotatable around the longitudinal axis A of the tubular column. The composition of continuous aqueous phase 4 from reservoir 16 may be the same as the continuous aqueous phase from reservoir 13.

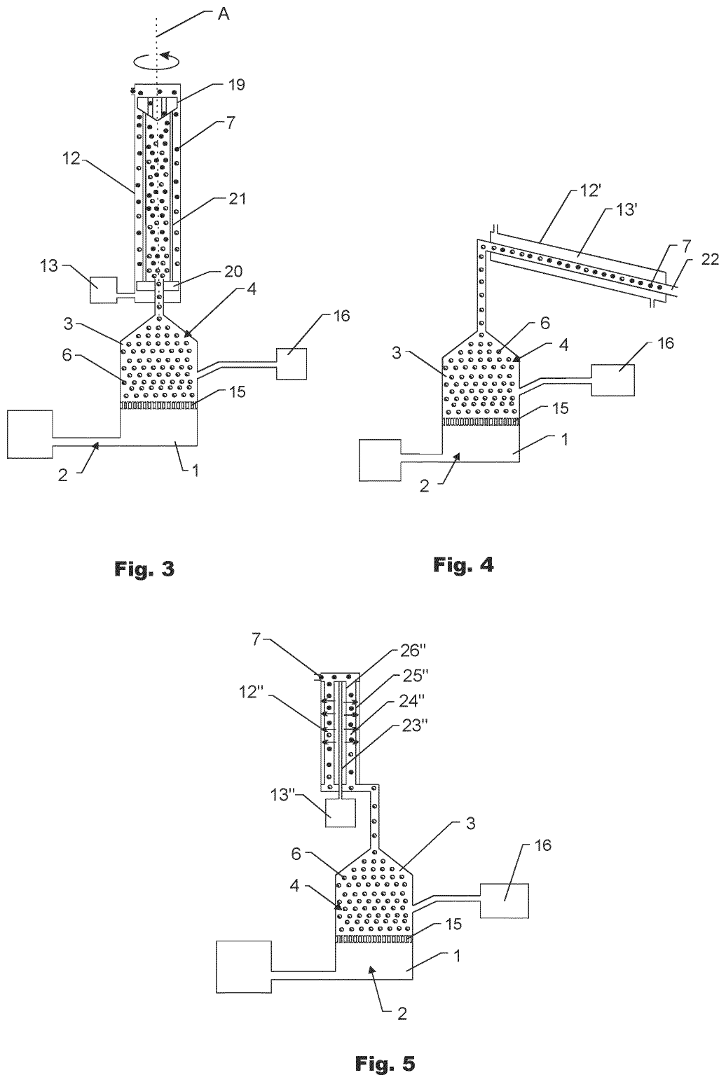

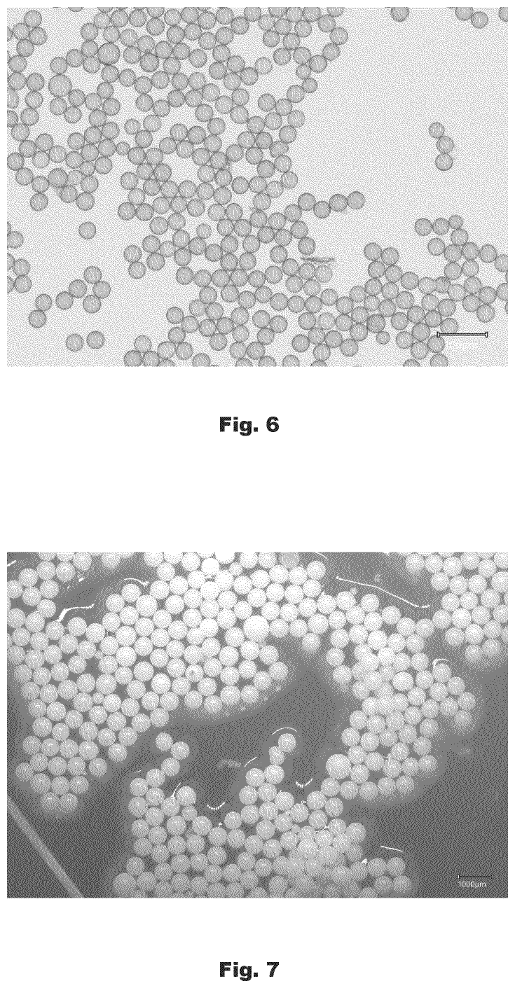

FIG. 4 shows schematically another embodiment of the method according to the invention. Again, the emulsion or dispersion 6 of droplet phase 2 in continuous aqueous phase 4 is generated in a similar manner as in the embodiment of FIG. 1 and as it is the case for the embodiment shown in FIG. 3, the cooling step is not conducted as a batch process but continuously using a cooling column 12′. In contrast to the embodiment of FIG. 3 however, cooling column 12′ does not physically mix emulsion/dispersion 6 with the cooling medium 13′ which may be water, but using a cooling column 12′ which provides two fluidic separated flow paths, i.e. inner flow path 22 for guiding emulsion/dispersion 6 through cooling column 12′ and outer flow path 13′ which circumferentially surrounds inner flow path 22. As can be seen, inner flow path 22 and outer flow path 13′ are coaxial to each other. Cooling column 12′ may be a condenser. A cooling medium is continuously provided via outer flow path 13′ and flows through the through the outer flow path. Such a cooling medium has a temperature below the transition temperature of the hydrophobic matrix such that it withdraws thermal energy from emulsions/dispersion 6 and in particular from the liquid hydrophobic matrix thereby inducing solidification of the hydrophobic matrix and forming capsules with solid matrix 7.

FIG. 5 shows schematically another embodiment of the method according to the invention. Again, the emulsion or dispersion 6 of droplet phase 2 in continuous aqueous phase 4 is generated in a similar manner as in the embodiment of FIG. 1 and as it is the case for the embodiment shown in FIG. 3, the cooling step is not conducted as a batch process but continuously using a cooling column 12″ which comprises, respectively is, a tubular column. As it is the case for the embodiment shown in FIG. 3, also in this embodiment, the cooling column 12″ does physically mix emulsion/dispersion 6 with the cooling medium 13′, which in this embodiment is also referred to as cross-flow fluid. The emulsion/dispersion 6 is removed from second chamber 3 via a dispersion outlet and introduced into longitudinally arranged dispersion channel 24″ of cooling column 12″ via a dispersion channel inlet. The emulsion or dispersion of the droplet phase in the continuous aqueous phase is transported through the dispersion channel 24″ along the longitudinal direction LO of the tubular column through the tubular column. The tubular column further comprises first mesh unit 25″ and a cross-flow fluid inlet unit 23″ which is connected to a cross-flow fluid reservoir 13″. The cross-flow fluid inlet unit 23″ introduces a cross-flow fluid into the dispersion channel in such a way that the introduced cross-flow fluid flows transversely, and in particular perpendicularly, to the longitudinal direction of the tubular column (see horizontal, i.e. radial, arrows). The cross-flow fluid inlet unit 23″ is configured such that the cross flow fluid flows through the first mesh unit 25″. Since the cross-flow fluid has a temperature which is lower than the first and/or second operating temperature and in particular which is lower than the transition temperature or the storage temperature of the hydrophobic matrix, the hydrophobic matrix solidifies within dispersion channel 24″ thereby forming capsules with solid matrix 7. These may generally be collected via a dispersion channel outlet. Typically, and in general, the first mesh unit may be fluidic connected to a collecting channel through which the cross-flow fluid and also any removed continuous aqueous phase can be collected and withdrawn from the cooling column. The cross-flow fluid inlet unit 23″ comprises second mesh unit 26″ which introduces the cross-flow fluid into the dispersion channel 24″. The second mesh unit introduces the cross-flow fluid transversely, in particular perpendicularly, to the longitudinal direction of the tubular column, respectively the dispersion channel 24″ (see horizontal, i.e. radial, arrows). As can be seen, first mesh unit 25″ and second mesh unit 26″ are radially spaced apart from each other thereby forming dispersion channel 24″ between them.

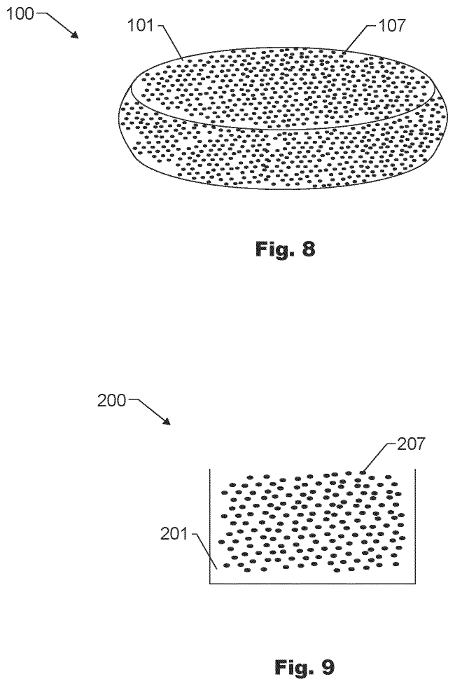

FIG. 8 shows food product 100, which in this embodiment is a meat substitute food product in the form of a burger patty. The food product 100 comprises an assembly of capsules 107 being mixed with other ingredients 101, such as a pea matrix or soy matrix. It is noted that the capsule size is illustrated as an exaggeration.

FIG. 9 shows a cosmetic product 200 which in this embodiment is a skin care product, which comprises an assembly of capsules 207 being combined with lotion 201.

### EXAMPLES

### Example 1 for a Composition Encapsulating a Hydrophobic Compound of Interest or a Powder as Compound of Interest

A mixture of GV 38/40 (palm kernel butter, CAS: 84540-04-5, 60 wt %) and VGB 22 (High Erucic Rapeseed Wax, 20 wt %) is used as liquid hydrophobic matrix in which 20 wt % of an hydrophobic compound of interest is dissolved, respectively in which 20 wt % of a powder as compound of interest is suspended at the first operating temperature of 60° C., thereby forming the droplet phase. Then, step emulsification according to steps a. to c. of the method of the invention is performed at a rate of 2 mL/min with the droplet phase as described above and water as the continuous aqueous phase, which further comprises PVA (CAS: 9002-89-5, 1 wt %) as a first surfactant. The pressure with which the droplet phase is introduced into the first chamber is set to 100 mbar above atmospheric pressure and the pressure with which the continuous aqueous phase is introduced into the second chamber is set to 150 mbar above atmospheric pressure. The thereby generated emulsion/dispersion of the droplet phase in the continuous aqueous phase is guided into a batch reactor comprising cooling water having a temperature of 20° C. under stirring with >500 rpm. The thereby generated capsules having a solid matrix are then separated from the water and the continuous aqueous phase by sieving and then dried under sub-atmospheric pressure.

### Example 2 for a Composition Encapsulating a Hydrophilic Compound of Interest

At first, an aqueous droplet phase is produced by mixing water (35 wt % of the resulting droplet phase) with a hydrophilic compound of interest, for example caffeine, (5 wt % of the resulting droplet phase). This aqueous droplet phase is then added at the first operating temperature of 70° C. to a mixture of 59 wt % of VGB 22 (High Erucic Rapeseed Wax) as liquid hydrophobic matrix and 1 wt % of PGPR 4150 as surfactant. Upon mixing at 10 000 rpm for 5 min, the droplet phase is formed which is an emulsion of the aqueous droplet phase in the liquid hydrophobic matrix. Then, a step emulsification according to steps a. to c. of the method of the invention is performed at a rate of 2 mL/min with the droplet phase as described and above and water as the continuous phase, which further comprises PVA (CAS: 9002-89-5, 1 wt %) as a first surfactant. The pressure with which the droplet phase is introduced into the first chamber is set to 100 mbar above atmospheric pressure and the pressure with which the continuous aqueous phase is introduced into the second chamber is set to 150 mbar above atmospheric pressure. The thereby generated emulsion/dispersion of the droplet phase in the continuous aqueous phase is guided into a batch reactor comprising cooling water having a temperature of 20° C. under stirring with >1000 rpm. The thereby generated capsules having a solid matrix are then separated from the water and the continuous aqueous phase by sieving and then dried under sub-atmospheric pressure.

1. Method for generating capsules with a solid matrix (7), the method comprising the steps:
a. Providing in a first chamber (1) a droplet phase (2) at a first operating temperature, the droplet phase (2) comprising a hydrophobic matrix, wherein the first temperature is selected such that the hydrophobic matrix is liquid and wherein the hydrophobic matrix is configured such that it is solid at a storage temperature, wherein the first operating temperature is higher than the storage temperature;
b. Providing in a second chamber (3) a continuous aqueous phase (4) at a second operating temperature, the continuous aqueous phase (4) comprising water and optionally at least one first surfactant;
wherein the first chamber (1) and the second chamber (3) are fluidic connected by one or more channels (5); the method further comprising:
c. Guiding the droplet phase (2) from the first chamber (1) through the one or more channels (5) into the second chamber (3) to form an emulsion or a dispersion (6) of the droplet phase (2) in the continuous aqueous phase (4);
d. Cooling the formed emulsion or dispersion (6) of the droplet phase (2) in the continuous aqueous phase (4) below the first operating temperature such that the hydrophobic matrix solidifies to form a solid matrix thereby generating a capsule with a solid matrix (7).

2. The method according to claim 1, wherein the first operating temperature is in the range from 20° C. to 90° C.; and/or wherein the second operating temperature is in the range from 20° C. to 90° C.; and/or wherein the storage temperature is in the range of 40° C. or less.

3. The method according to claim 1, wherein the first operating temperature is the same or higher as the second operating temperature; and/or wherein the second temperature is equal or higher than the storage temperature.

4. The method according to claim 1, wherein the droplet phase (2) in step a. additionally comprises at least one compound of interest.

5. The method according to claim 4, wherein the at least one compound of interest is a powder being dispersed in the droplet phase (2).

6. The method according to claim 1, wherein the droplet phase (2) provided in the first chamber (1) in step a. is an emulsion (8) of an aqueous droplet phase (9) in the liquid hydrophobic matrix (10), wherein the droplet phase (2) further comprises at least one second surfactant.

7. The method according to claim 6, wherein the aqueous droplet phase (9) comprises at least one hydrophilic compound of interest.

8. The method according to claim 1, wherein step d. is performed in a cooling bath in a batch reactor (11), or wherein step d. is performed in a cooling column (12, 12′) by guiding the formed emulsion or dispersion (6) of the droplet phase (2) in the continuous aqueous phase (4) through the cooling column (12, 12′).

9. The method according to claim 8, wherein the cooling column (12, 12′) comprises one or more cooling elements being configured for cooling the emulsion or dispersion (6) of the droplet phase (2) in the continuous aqueous phase (4).

10. The method according to claim 8, wherein the formed emulsion or dispersion (6) of the droplet phase (2) in the continuous aqueous phase (4) is guided through the cooling column (12′) such that it is fluidic separated from a cooling medium (13′); and/or wherein the formed emulsion or dispersion (6) of the droplet phase (2) in the continuous aqueous phase (4) is guided through the cooling column (12) such that it is mixed with a cooling medium (13).

11. The method according to claim 1, wherein the liquid hydrophobic matrix comprises a wax, a fat and/or an oil.

12. The method according to claim 1, wherein the liquid hydrophobic matrix is configured such that it has a melting point at which 100% of the liquid hydrophobic matrix is liquid in the range of 20° C. to 90° C.; and/or wherein the liquid hydrophobic matrix is configured such that it has a melting starting point in the range of 15° C. to 85° C.

13. The method according to claim 1, wherein the liquid hydrophobic matrix is selected such that the melting range interval is between 5 K and 45 K.

14. Method for generating a food product comprising the method according to claim 1 and further comprising combining the generated capsule with a solid matrix with one or more ingredients of a food product to generate the food product.

15. Method for generating a cosmetic product comprising the method according to claim 1 and further comprising combining the generated capsule with a solid matrix with one or more cosmetic ingredients of a cosmetic product to generate the cosmetic product.

16. An assembly of capsules, particularly microcapsules, comprising a plurality of capsules (7) produced according to the method according to claim 1.

17. The assembly of capsules according to claim 16, wherein the capsules (7) have an equal size distribution with a coefficient of variation of 10% or less.

18. Use of an assembly of capsules according to claim 16 in a food product, in particular for food coloring, flavor incorporation and/or for increasing juiciness of the food product.

19. Use of an assembly according to claim 16 in a cosmetic product, in particular for as delivery vehicle and/or moisturizer.

20. Food product, in particular a meat substitute food product, comprising the assembly of capsules according to claim 16.

21. Cosmetic product, in particular skin care product, comprising the assembly of capsules according to claim 16.

22. The method according to claim 10, wherein the cooling medium (13) is water.

23. The method according to claim 11, wherein the liquid hydrophobic matrix comprises a hydrogenated oil, a vegetarian oil, such as rapeseed oil, palm kernel oil, sunflower seed oil, hemp oil, canola oil, palm oil, soybean oil, coconut oil, olive oil.
